(19) United States
(12) Patent Application Publication
(10) Pub. No.: US 2006/0058286 A1
(43) Pub. Date: Mar. 16, 2006

(54) Methods of treating HIV infection

(76) Inventors: Mark Krystal, Westport, CT (US); Carol A. Deminie, Cheshire, CT (US); Sagarika Bollini, Hamden, CT (US); Brian J. Terry, Middletown, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/227,526

(22) Filed: Sep. 15, 2005

Related U.S. Application Data
(60) Provisional application No. 60/610,343, filed on Sep. 16, 2004.

(51) Int. Cl. A61K 31/551; A61K 31/522; A61K 31/513; A61K 31/195

(52) U.S. Cl. 514/220; 514/563; 514/463; 514/263.31; 514/269

(57) ABSTRACT

The invention encompasses pharmaceutical compositions and methods for using Compound 1 or Compound 2 in combination with other agents for treating patients with AIDS or HIV infection.

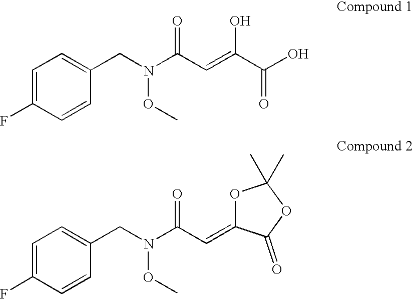

### REFERENCE TO RELATED APPLICATIONS

This application claims the benefit of U.S. Provisional Application Ser. No. 60/610,343 filed Sep. 16, 2004.

### BACKGROUND OF THE INVENTION

HIV-1 (human immunodeficiency virus -1) infection remains a major medical problem, with an estimated 42 million people infected worldwide at the end of 2002. The number of cases of HIV and AIDS (acquired immunodeficiency syndrome) has risen rapidly. In 2002, ˜5.0 million new infections were reported, and 3.1 million people died from AIDS. Currently available drugs for the treatment of HIV include ten nucleoside reverse transcriptase (RT) inhibitors or approved single pill combinations: zidovudine or AZT (or Retrovir®), didanosine or DDI (or Videx®), stavudine or D4T (or Zerit®), lamivudine or 3TC (or Epivir®), zalcitabine or DDC (or Hivid®), abacavir succinate (or Ziagen®), tenofovir disoproxil fumarate salt (or Viread®), emtricitabine (or Emtriva®), Combivir® (contains 3TC and AZT), Trizivir® (contains abacavir, 3TC and AZT), Truvada™ (contains tenofovir and emtricitabine), Epzicom™ (contains abacavir and 3TC); three non-nucleoside reverse transcriptase inhibitors: nevirapine (or Viramune®), delavirdine (or Rescriptor®) and efavirenz (or Sustiva®), eight peptidomimetic protease inhibitors or approved formulations: saquinavir (or Invirase® or Fortovase®), indinavir (or Crixivan®), ritonavir (or Norvir®), nelfinavir (or Viracept®), amprenavir (or Agenerase®), atazanavir (Reyataz®), fosamprenavir (or Lexiva), Kaletra®(contains lopinavir and ritonavir), and one fusion inhibitor enfuvirtide (or T-20 or Fuzeon®).

Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30% to 50% of patients ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present (Larder and Kemp; Gulick; Kuritzkes; Morris-Jones et al; Schinazi et al; Vacca and Condra; Flexner; Berkhout and Ren et al; (Ref. 6-14). Thus, there is continuing need for new compounds and methods of treatment for HIV infection.

(Z)-3-[(4-Fluoro-benzyl)-methoxy-carbamoyl]-2-hydroxy-acrylic acid (Compound 1) and 2-[2,2-Dimethyl-5-oxo-[1,3]dioxolan-(4Z)-ylidene]-N-(4-fluoro-benzyl)-N-methoxy-acetamide (Compound 2, a corresponding prodrug), are HIV-1 integrase inhibitors demonstrating potent antiviral activity against a variety of laboratory and clinical strains of HIV-1. Compound 1 and 2 were described in U.S. Pat. No. 6,777,440 which is herein incorporated by reference in its entirety.

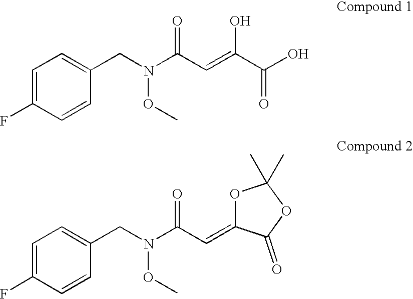

Compound 1((Z)-3-[(4-Fluoro-benzyl)-methoxy-carbamoyl]-2-hydroxy-acrylic acid and the corresponding prodrug) acts by selectively inhibiting the viral integrase enzyme. Integrase is required for the proviral DNA integration step of HIV infection. By inhibiting this enzyme, Compound 1 blocks the production of progeny viruses. Compound 2 (2-[2,2-Dimethyl-5-oxo-[1,3]dioxolan-(4Z)-ylidene]-N-(4-fluoro-benzyl)-N-methoxy-acetamide) is a prodrug of compound 1 and forms compound 1 in-vivo.

Compound 3, Compound 4, and Compound 5 are HIV attachment inhibitors described in U.S. Pat. No. 6,476,034, U.S. Pat. No. 6,632,819, and U.S. patent application Ser. No. US 2003 0207910, published Nov. 6, 2003.

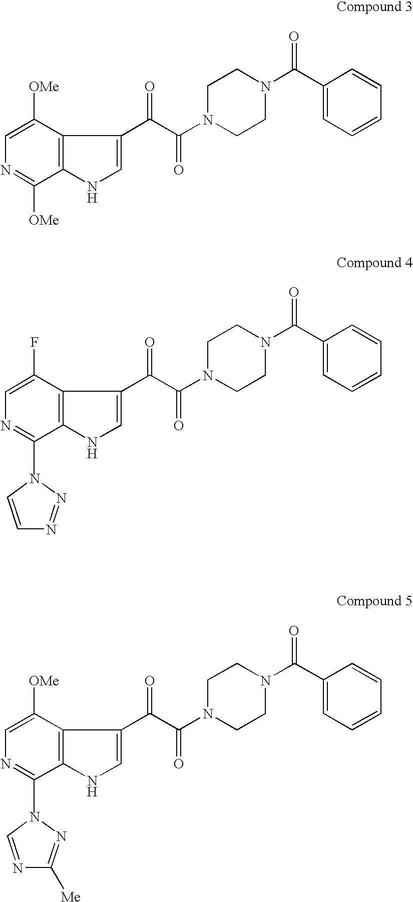

### DESCRIPTION OF THE INVENTION

The invention encompasses pharmaceutical compositions and methods for treating patients infected with the HIV virus.

One aspect of the invention is a method for treating HIV infection in a human patient comprising the administration of a therapeutically effective amount of 3-[(4-fluorobenzyl)methoxycarbamoyl]-2-hydroxyacrylic acid (Compound 1) or 2-(2,2)-dimethyl-5-oxo-[1,3]-dioxolan-4-ylidene)-N-(4-fluorobenzyl)-N-methoxyacetamide (Compound 2) or a pharmaceutically acceptable salt, or solvate thereof with a therapeutically effective amount of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

Another aspect of the invention is a method wherein the agent is a nucleoside HIV reverse transcriptase inhibitor.

Another aspect of the invention is a method wherein the nucleoside HIV reverse transcriptase inhibitor is selected from the group consisting of abacavir, didanosine, emtricitabine, lamivudine, stavudine, tenofovir, zalcitabine, zidovudine, tenofovir disproxil fumarate, emtricitabine, enfuvirtide, lamivudine, Combivir® and Trizivir® or a pharmaceutically acceptable salt or solvate thereof.

Another aspect of the invention is a method wherein the agent is a non-nucleoside HIV reverse transcriptase inhibitor.

Another aspect of the invention is a method wherein the non-nucleoside HIV reverse transcriptase inhibitor is selected from the group consisting of delavirdine, efavirenz, and nevirapine, or a pharmaceutically acceptable salt or solvate thereof.

Another aspect of the invention is a method wherein the agent is an HIV protease inhibitor.

Another aspect of the invention is a method wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

Another aspect of the invention is a method wherein the agent is an HIV fusion inhibitor.

Another aspect of the invention is a method wherein the HIV fusion inhibitor is enfuvirtide or T-1249, or a pharmaceutically acceptable salt or solvate thereof.

Another aspect of the invention is a method wherein the agent is an HIV attachment inhibitor.

Another aspect of the invention is a method wherein the HIV attachment inhibitor is Compound 3, Compound 4, or Compound 5 or a pharmaceutically acceptable salt or solvate thereof.

Another aspect of the invention is a method wherein the agent is a CCR5 inhibitor.

Another aspect of the invention is a method wherein the CCR5 inhibitor is selected from the group consisting of Sch-C, Sch-D, TAK-220, PRO-140, and UK-427,857, or their analogs, or a pharmaceutically acceptable salt or solvate thereof

Another aspect of the invention is a method wherein the agent is a CXCR4 inhibitor.

Another aspect of the invention is a method wherein the CXCR4 inhibitor is AMD-3100 or its analogs, or a pharmaceutically acceptable salt or solvate thereof.

Another aspect of the invention is a method wherein the agent is an HIV budding or maturation inhibitor.

Another aspect of the invention is a method wherein the budding or maturation inhibitor is PA-457 or its analogs, or a pharmaceutically acceptable salt, or solvate thereof.

Another aspect of the invention is a method wherein the agent is an HIV integrase inhibitor.

Another aspect of the invention is a method wherein the integrase inhibitor is C-2507 or its analogs, L-870810 or its analogs, L-870812 or its analogs, 1380 or its analogs, and JTK-303 or its analogs.

Another aspect of the invention is a pharmaceutical composition useful for treating AIDS or HIV infection comprising a therapeutically effective amount 3-[(4-fluorobenzyl)methoxycarbamoyl]-2-hydroxyacrylic acid or 2-(2,2)-dimethyl-5-oxo-[1,3]-dioxolan-4-ylidene)-N-(4-fluorobenzyl)-N-methoxyacetamide or a pharmaceutically acceptable salt, or solvate thereof with at least one other agent used for treatment of AIDS, or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

Another aspect of the invention is the composition wherein the agent is a nucleoside HIV reverse transcriptase inhibitor.

Another aspect of the invention is the composition wherein the nucleoside HIV transcriptase inhibitor is selected from the group consisting of abacavir, didanosine, emtricitabine, lamivudine, stavudine, tenofovir, zalcitabine, and zidovudine, tenofovir disproxil fumarate, emtricitabine, enfuvirtide, lamivudine, Combivir® and Trizivir® or a pharmaceutically acceptable salt or solvate thereof.

Another aspect of the invention is the composition wherein the agent is a non-nucleoside HIV reverse transcriptase inhibitor.

Another aspect of the invention is the composition wherein the non-nucleoside HIV reverse transcriptase inhibitor is selected from the group consisting of delavirdine, efavirenz, and nevirapine, or a pharmaceutically acceptable salt or solvate thereof.

Another aspect of the invention is the composition wherein the agent is an HIV protease inhibitor.

Another aspect of the invention is the composition wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

Another aspect of the invention is the composition wherein the agent is an HIV fusion inhibitor.

Another aspect of the invention is the composition method wherein the HIV fusion inhibitor is enfuvirtide or T-1249, or a pharmaceutically acceptable salt or solvate thereof.

Another aspect of the invention is the composition wherein the agent is an HIV attachment inhibitor.

Another aspect of the invention is the composition wherein the HIV attachment inhibitor is Compound 3, Compound 4, or Compound 5.

Another aspect of the invention is the composition wherein the agent is a CCR5 inhibitor.

Another aspect of the invention is the composition wherein the CCR5 inhibitor is selected from the group consisting of Sch-C, Sch-D, TAK-220, PRO-140, and UK-427,857, or a pharmaceutically acceptable salt or solvate thereof.

Another aspect of the invention is a method wherein the agent is a CXCR4 inhibitor.

Another aspect of the invention is a method wherein the CXCR4 inhibitor is AMD-3100 or a pharmaceutically acceptable salt or solvate thereof.

Another aspect of the invention is the composition wherein the agent is an HIV budding or maturation inhibitor.

Another aspect of the invention is the composition wherein the budding or maturation inhibitor is PA-457, or a pharmaceutically acceptable salt or solvate thereof.

Another aspect of the invention is the composition wherein the agent is an HIV integrase inhibitor.

“Combination,” “coadministration,” “concurrent,” and similar terms referring to the administration of Compound 1 with at least one anti-HIV agent mean that the components are part of a combination antiretroviral therapy or highly active antiretroviral therapy (HAART) as understood by practitioners in the field of AIDS and HIV infection.

“Therapeutically effective” means the amount of agent required to provide a meaningful patient benefit as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus and suitable for therapy as understood by practitioners in the field of AIDS and HIV infection.

“Treatment,” “therapy,” “regimen,” “HIV infection,” “ARC,” “AIDS” and related terms are used as understood by practitioners in the field of AIDS and HIV infection.

The invention includes all pharmaceutically acceptable salt forms of Compound 1. Pharmaceutically acceptable salts are those in which the counter ions do not contribute significantly to the physiological activity or toxicity of the compounds and as such function as pharmacological equivalents. In many instances, salts have physical properties that make them desirable for formulation, such as solubility or crystallinity. The salts can be made according to common organic techniques employing commercially available reagents. Suitable anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate.

The invention also includes all solvated forms of Compound 1, particularly hydrates. Solvates do not contribute significantly to the physiological activity or toxicity of the compounds and as such function as pharmacological equivalents. Solvates may form in stoichiometric amounts or may form from adventitious solvent or a combination of both. One type of solvate is hydrate. Some hydrated forms include monohydrate, hemihydrate, and dihydrate.

### Biological Methods

Compound 1 demonstrated synergistic or additive-synergistic HIV antiviral activity when used in conjunction with a variety of other antiviral agents, as described below.

Virus and cell lines. The T-cell line, MT-2 was obtained through the AIDS Research and Reference Reagent Program, NIAID and was contributed by Dr. D. Richman. The cell line was cultured in RPMI 1640 medium supplemented with 10% fetal bovine serum, 2 mM L-glutamine and sub-cultured twice a week. The NL4-3 strain of HIV-1 was obtained from the AIDS Research and Reference Reagent Program. Both virus stocks were amplified and titered in MT-2 cells using a virus infectivity assay.

Chemicals. Compound 1, atazanavir, didanosine, stavudine, efavirenz, enfuvirtide (T-20), and Compound 3 were synthesized by Bristol-Myers Squibb using published or known reactions. Amprenavir, indinavir, nelfinavir, nevirapine, lopinavir, lamivudine, ritonavir, tenofovir, saquinavir, delavirdine and abacavir were extracted from commercial formulations of the prescribed drugs and purified using published or common techniques. Tenofovir was tested as tenovir disopoxil fumerate. Zalcitabine was obtained from the National Institutes of Health. Zidovudine was purchased from Sigma, and emtricitabine—from Moravek Biochemicals.

Drug Susceptibility and Cytotoxicity Assays. For drug susceptibility assays, MT-2 cells were infected with HIV-1 NL4-3 at an MOI of 0.001, and seeded into 96-well microtiter plates (2.5×10⁵ cells/ml) containing serial dilutions of test compounds. The drug combinations were set up using ratios of the two drugs of 1:1, 1:2.5 and 2.5:1 times the EC₅₀ value determined for each drug in prior multiple experiments. Each drug ratio consisted of an array of 3-fold serial dilutions, and was performed in quadruplicate. The plates were incubated at 37° C./5% CO₂. The MT-2 cells infected with HIV-1 NL4-3 were incubated for 5 days. On day-five post-infection, 20 μl from each well was harvested and quantitated by a reverse transcriptase (RT) assay or an MTS assay. Cytotoxicity assays were performed using uninfected cells, exposed to the same drug combinations, and incubated for five days. Cell viability was determined by an XTT assay. The CC₅₀ values were calculated by using the exponential form of the median effect equation as mentioned below for calculation of EC₅₀.

Analysis of Drug Combination Effects. For determination of CI values, drugs were diluted in a fixed ratio and multiple ratios were analyzed. The drug serial dilutions spanned a range of concentrations near the EC₅₀ value of each compound, so that equivalent antiviral activities could be compared. Concentration-response curves were estimated for each individual drug and every combination using the median-effect equation. The equation was fit using a nonlinear regression routine (Proc Nlin) in PC SAS version 8.01 (SAS Institute Inc., SAS Version 8.01, Cary, N.C.: SAS Institute Inc., 1990).

EC₅₀ values for each drug were determined from the single drug experiments, using the median effect equation, Fa=1/[+(ED₅₀/drug concentration)^{m}]. In this equation, Fa stands for “fraction affected,” and represents the fraction of the viral load that has been inactivated. For example, Fa of 0.75 indicates that viral replication had been inhibited by 75%, relative to the no-drug controls. ED₅₀ is drug concentration that is expected to reduce the amount of virus by 50%, and m is a parameter that reflects the slope of the concentration-response curve.

To assess antiviral effects of different drug combination treatments, combination indices (CIs) were calculated according to Chou and Rideout. The combination index was computed as
CI=[D]_{1/[Dm]1}+[D]_{2/[Dm]2}

In this equation [Dm]1 and [Dm]2 are the concentrations of drugs that would individually produce a specific level of effect, while [D]1 and [D]2 are the concentrations of drugs in combination that would produce the same level of effect.

Theoretically, additivity is implied if the CI is equal to one, synergy if the CI is less than one, and antagonism if the CI is greater than one. However, extensive experience with combination studies indicates that there are inherent laboratory variables that must be taken into account in interpreting the CIs. At best, we can construct a range that contains the likely values for the CI, given the noise in the data. In this report, these ranges are reported in parentheses next to each point estimate of the CI. For example, when we report a CI of “0.53 (0.46, 0.60)” this means that our best estimate of the CI is 0.53, but due to noise in the data, values from 0.46 to 0.60 are also reasonable values for the CI. This range, 0.46 to 0.60 falls entirely below the value of 1.0, and hence all likely values for the CI are less than 1.0. Therefore, we can infer synergistic behavior for this case. If the range fell entirely above 1.0, we would infer antagonistic behavior. If the range were to include 1.0, we would infer additivity.

In carrying out the combination experiments below, the EC₅₀ for Compound 1 and each comparator compound was determined during the course of each study, and used in the subsequent data analysis. The determined values are consistent with our previously published data and are shown in Table 1.

TABLE 1
Anti-HIV Activity of the Compounds Used in Two-Drug Combination
Studies

| | | Highest Conc.Used |
|---|---|---|
| Compound | EC₅₀ (μM) | (μM) |
| Compound 1 | 0.155 | 250 |
| Abacavir | 0.74 | 500 |
| Tenofovir | 0.003 | 5.0 |
| Zalcitabine | 0.12 | 125 |
| Didanosine | 0.27 | 250 |
| Stavudine | 0.055 | 250 |
| Zidovudine | 0.002 | 12.5 |
| Lamivudine | 0.19 | 250 |
| Emtricitabine | 0.11 | 125 |
| Efavirenz | 0.0015 | 0.25 |
| Nevirapine | 0.085 | 50 |
| Delavirdine | 0.050 | 25 |
| Indinavir | 0.006 | 25 |
| Atazanavir | 0.009 | 12.5 |
| Lopinavir | 0.014 | 5 |
| Nelfinavir | 0.008 | 25 |
| Amprenavir | 0.042 | 12.5 |
| Saquinavir | 0.011 | 12.5 |
| Ritonavir | 0.024 | 50 |
| Enfuvirtide | 0.007 | 0.555 |
| Compound 3 | 0.001 | 2.25 |

Two-Drug Combinations of Compound 1 with Nucleoside Reverse Transcriptase Inhibitors. Eight nucleoside RT inhibitors (didanosine, stavudine, zidovudine, lamivudine, abacavir, zalcitabine, emtricitibine and the nucleoside phosphonate tenofovir) were combined with Compound 1 at a range of concentrations near the EC₅₀ value of each compound, so that equivalent antiviral activities could be compared. All estimates were computed using SAS Proc NLIN, and a two-parameter logistic. Data is presented in Table 2 as the combination indices and the asymptotic confidence intervals for RT inhibitors at different molar ratios (see Materials and Methods).

Four nucleoside RT inhibitors; zidovudine, didanosine, zalcitabine, and emtricitibine, show synergistic antiviral effects in combination with Compound 1 at all effective levels and all molar ratios. Stavudine exhibits synergy at the 75% and 90% effective levels and additivity at the 50% effective level. Lamivudine exhibits synergy at the 1:1 and 0.04:1 molar ratios, with a bias toward additivity at the 2.5:1 molar ratio. The overall effects of stavudine and lamivudine are therefore classified as additive-synergistic. Abacavir exhibits additivity at the 75% and 90% effective levels and synergy at the 50% effective levels, for all three molar ratios tested. Tenofovir exhibits additivity at all molar ratios and all effective levels. The overall effects of the latter two compounds are therefore classified as additive. Taking all the CI values and the analyses into account, the overall effect of combining nucleoside RT inhibitors with Compound 1 is in the range of additive to synergistic. No significant antagonism of anti-HIV activity is observed. No enhanced cytotoxicity was encountered at the highest concentrations tested with any of the drug combinations, as measured by XTT reduction assay.

TABLE 2
Two-Drug Combinations using Compound 1 and Nucleoside Reverse
Transcriptase Inhibitors.

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |
| Zidovudine | | | | |
| 10:1 (1:1) | 0.21 (0.16, 0.26) | 0.15 (0.10, 0.20) | 0.12 (0.06, 0.18) | Synergistic |
| 4:1 (1:2.5) | 0.33 (0.26, 0.40) | 0.23 (0.16, 0.29) | 0.16 (0.09, 0.24) | |
| 25:1 (2.5:1) | 0.21 (0.18, 0.24) | 0.16 (0.12, 0.19) | 0.13 (0.08, 0.17) | |
| Didanosine | | | | |
| 0.5:1 (1:1) | 0.20 (0.14, 0.25) | 0.27 (0.17, 0.37) | 0.40 (0.16, 0.64) | Synergistic |
| 0.2:1 (1:2.5) | 0.16 (0.12, 0.20) | 0.21 (0.14, 0.28) | 0.31 (0.13, 0.49) | |
| 1.25:1 (2.5:1) | 0.31 (0.27, 0.36) | 0.32 (0.26, 0.39) | 0.35 (0.23, 0.47) | |
| Stavudine | | | | |
| 1:1 (1:1) | 0.63 (0.42, 0.85) | 0.51 (0.29, 0.74) | 0.43 (0.10, 0.75) | Additive- |
| 0.4:1 (1:2.5) | 1.32 (0.94, 1.69) | 0.68 (0.41, 0.94) | 0.36 (0.12, 0.59) | Synergistic |
| 2.5:1 (2.5:1) | 1.20 (0.88, 1.51) | 0.71 (0.45, 0.96) | 0.43 (0.17, 0.68) | |
| Lamivudine | | | | |
| 1:1 (1:1) | 0.69 (0.58, 0.81) | 0.49 (0.38, 0.61) | 0.37 (0.25, 0.50) | Additive- |
| 0.4:1 (1:2.5) | 0.58 (0.52, 0.65) | 0.60 (0.51, 0.69) | 0.64 (0.49, 0.80) | Synergistic |
| 2.5:1 (2.5:1) | 0.93 (0.65, 1.20) | 0.78 (0.46, 1.10) | 0.70 (0.24, 1.16) | |
| Abacavir | | | | |
| 0.5:1 (1:1) | 0.66 (0.48, 0.84) | 0.89 (0.55, 1.91) | 1.20 (0.48, 1.91) | Additive |
| 0.2:1 (1:2.5) | 0.58 (0.45, 0.71) | 0.92 (0.62, 1.21) | 1.46 (0.72, 2.20) | |
| 1.25:1 (2.5:1) | 0.65 (0.44, 0.86) | 0.88 (0.49, 1.28) | 1.20 (0.36, 2.05) | |
| Tenofovir | | | | |
| 50:1 (1:1) | 1.30 (1.00, 1.61) | 1.02 (0.69, 1.35) | 0.80 (0.39, 1.21) | Additive |
| 20:1 (1:2.5) | 0.93 (0.63, 1.23) | 0.99 (0.54, 1.43) | 1.06 (0.31, 1.80) | |
| 125:1 (2.5:1) | 1.03 (0.65, 1.40) | 0.84 (0.42, 1.27) | 0.70 (0.14, 1.25) | |
| Zalcitabine | | | | |
| 2:1 (1:1) | 0.79 (0.64, 0.94) | 0.75 (0.55, 0.96) | 0.72 (0.42, 1.02) | Synergistic |
| 4:5 (1:2.5) | 0.83 (0.70, 0.95) | 0.70 (0.56, 0.85) | 0.60 (0.41, 0.80) | |
| 5:1 (2.5:1) | 0.85 (0.75, 0.95) | 0.72 (0.61, 0.84) | 0.61 (0.46, 0.77) | |
| Emtricitabine | | | | |
| 1:1 (1:1) | 0.52 (0.44, 0.59) | 0.40 (0.32, 0.48) | 0.32 (0.22, 0.41) | Synergistic |
| 0.4:1 (1:2.5) | 0.74 (0.63, 0.84) | 0.64 (0.52, 0.77) | 0.57 (0.39, 0.75) | |
| 2.5:1 (2.5:1) | 0.49 (0.42, 0.56) | 0.34 (0.28, 0.41) | 0.25 (0.17, 0.33) | |

^{a}Ratio of Compound 1 (BMS-538158) to comparator compound
^{b}A lower bound of the asymptotic confidence interval greater than 1 indicates antagonisms, an upper bound of less than 1 indicates synergism and a value of 1 being contained in the interval indicates additivity. The 95% confidence intervals are shown in parenthesis, and represent a measure of variability in the data.

Two-Drug Combinations of Compound 1 with Non-Nucleoside Reverse Transcriptase Inhibitors. Three non-nucleoside RT inhibitors were combined with Compound 1 at a range of concentrations near the EC₅₀ value of each compound, as described above for nucleoside RT inhibitors. Data is presented in Table 3 as the combination indices and the asymptotic confidence intervals at different molar ratios. All three compounds, efavirenz, nevirapine, and delavirdine show strong synergistic effects in combination with Compound 1. Synergy is seen at all effective concentrations and at all molar ratios. No enhanced cytotoxicity was observed at the highest concentrations tested with any of the drug combinations, suggesting a potential for therapeutic efficacy of Compound 1 combinations with non-nucleoside RT inhibitors.

TABLE 3
Two-Drug Combinations using Compound 1 and Non-Nucleoside
ReverseTranscriptase Inhibitors

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |
| Efavirenz | | | | |
| 1000:1 (1:1) | 0.57 (0.44, 0.70) | 0.56 (0.38, 0.74) | 0.56 (0.28, 0.84) | Synergistic |
| 400:1 (1:2.5) | 0.49 (0.36, 0.62) | 0.54 (0.34, 0.75) | 0.61 (0.26, 0.97) | |
| 2500:1 (2.5:1) | 0.60 (0.45, 0.74) | 0.51 (0.33, 0.68) | 0.43 (0.20, 0.67) | |
| Nevirapine | | | | |
| 5:1 (1:1) | 0.51 (0.39, 0.63) | 0.30 (0.20, 0.40) | 0.18 (0.08, 0.27) | Synergistic |
| 2:1 (1:2.5) | 0.63 (0.42, 0.83) | 0.40 (0.22, 0.59) | 0.26 (0.07, 0.46) | |
| 12.5:1 (2.5:1) | 0.52 (0.39, 0.64) | 0.32 (0.22, 0.43) | 0.21 (0.10, 0.31) | |
| Delavirdine | | | | |
| 10:1 (1:1) | 0.64 (0.50, 0.78) | 0.59 (0.41, 0.78) | 0.58 (0.30, 0.85) | Synergistic |
| 4:1 (1:2.5) | 0.56 (0.47, 0.64) | 0.41 (0.32, 0.50) | 0.31 (0.20, 0.41) | |
| 25:1 (2.5:1) | 0.52 (0.44, 0.60) | 0.44 (0.34, 0.54) | 0.39 (0.26, 0.53) | |

^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

Two-Drug Combinations Involving Compound I and HIV Protease Inhibitors. Evaluation of Compound 1 for drug combination therapy with protease inhibitors was carried out using indinavir, amprenavir, nelfinavir, lopinavir, saquinavir, ritonavir and atazanavir. Results from this two-drug combination study are summarized in Table 4 and suggest additive to synergistic results using indinavir, amprenavir, lopinavir, saquinavir, and atazanavir. Nelfinavir exhibits synergy at the 5:1 and 2:1 molar ratios at all effective levels, with a bias toward additivity at the 12.5:1 molar ratio, but only t the 75% and 90% effective levels. The overall effect of nelfinavir was therefore classified as moderately synergistic. Ritonavir exhibits additive interactions at all molar ratios and all effective levels. No cytotoxicity was observed at the highest concentrations used in any of these combination antiviral assays.

TABLE 4
Two-Drug Combination using Compound 1 and Protease Inhibitors

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |
| Indinavir | | | | |
| 5:1 (1:1) | 0.64 (0.42, 0.85) | 1.17 (0.63, 1.71) | 2.15 (0.53, 3.78) | Additive- |
| 2:1 (1:2.5) | 0.92 (0.68, 1.16) | 1.07 (0.68, 1.46) | 1.24 (0.51, 1.97) | Synergistic |
| 12.5:1 (2.5:1) | 0.41 (0.29, 0.53) | 0.70 (0.42, 0.97) | 1.19 (0.40, 1.97) | |
| Nelfinavir | | | | |
| 5:1 (1:1) | 0.55 (0.45, 0.65) | 0.57 (0.42, 0.71) | 0.59 (0.35, 0.83) | Moderately |
| 2:1 (1:2.5) | 0.41 (0.34, 0.49) | 0.50 (0.38, 0.63) | 0.62 (0.36, 0.87) | Synergistic |
| 12.5:1 (2.5:1) | 0.82 (0.65, 0.99) | 0.84 (0.60, 1.08) | 0.87 (0.47, 1.27) | |
| Saquinavir | | | | |
| 20:1 (1:1) | 0.65 (0.49, 0.80) | 0.85 (0.56, 1.14) | 1.20 (0.55, 1.84) | Additive- |
| 8:1 (1:2.5) | 0.55 (0.42, 0.68) | 0.97 (0.65, 1.29) | 1.81 (0.86, 2.77) | Synergistic |
| 50:1 (2.5:1) | 0.47 (0.38, 0.56) | 0.60 (0.44, 0.75) | 0.82 (0.49, 1.15) | |
| Amprenavir | | | | |
| 20:1 (1:1) | 0.37 (0.24, 0.49) | 0.53 (0.28, 0.78) | 0.95 (0.24, 1.67) | Additive- |
| 8:1 (1:2.5) | 0.37 (0.27, 0.48) | 0.56 (0.33, 0.78) | 1.05 (0.38, 1.71) | Synergistic |
| 50:1 (2.5:1) | 1.17 (0.91, 1.43) | 0.87 (0.60, 1.13) | 0.74 (0.38, 1.10) | |
| Atazanavir | | | | |
| 20:1 (1:1) | 0.64 (0.54, 0.73) | 0.60 (0.49, 0.72) | 0.59 (0.43, 0.75) | Additive- |
| 8:1 (1:2.5) | 0.91 (0.81, 1.20) | 0.90 (0.70 1.10) | 0.90 (0.57, 1.23) | Synergistic |
| 50:1 (2.5:1) | 1.01 (0.80, 1.19) | 1.02 (0.77, 1.28) | 1.09 (0.67, 1.50) | |
| Lopinavir | | | | |
| 50:1 (1:1) | 0.16 (0.11, 0.22) | 0.16 (0.08, 0.24) | 0.16 (0.04, 0.28) | Additive- |
| 20:1 (1:2.5) | 1.04 (0.90, 1.18) | 0.57 (0.46 0.68) | 0.32 (0.22, 0.42) | Synergistic |
| 125:1 (2.5:1) | 1.60 (1.28, 1.92) | 0.78 (0.56, 1.00) | 0.38 (0.22, 0.55) | |
| Ritonavir | | | | |
| 5:1 (1:1) | 1.35 (0.72, 1.97) | 1.74 (0.61, 2.87) | 2.56 (0.00, 5.18) | Additive |
| 2:1 (1:2.5) | 1.23 (0.73, 1.74) | 1.24 (0.53, 1.96) | 1.39 (0.13, 2.65) | |
| 12.5:1 (2.5:1) | 1.37 (0.95, 1.80) | 1.18 (0.67, 1.68) | 1.13 (0.37, 1.88) | |

^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

Two-Drug Combination of Compound 1 with Entry Inhibitors. Enfuvirtide (T-20) is a recently approved HIV gp41 fusion inhibitor and the first approved Entry class inhibitor. The results presented in Table 5 indicate that the combination of Compound 1 with T-20 is synergistic to additive. Compound 3 represents a new class of HIV attachment inhibitors. Compound 3 shows moderate synergy at the 13:1 and 82.5:1 molar ratios and additivity at the 33:1 molar ratio. The overall effect is therefore classified as synergistic to additive. No significant cytotoxicity was observed at the highest concentration of the combined drugs.

TABLE 5
Anti-HIV Activity from a Two-Drug Combination using Compound 1
and Entry Inhibitors.

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |
| Enfuvirtide | | | | |
| 450:1 (1:1) | 0.74 (0.58, 0.89) | 0.95 (0.67, 1.24) | 1.25 (0.66, 1.83) | Additive- |
| 180:1 (1:2.5) | 0.72 (0.55, 0.89) | 0.69 (0.46, 0.91) | 0.68 (0.33, 1.02) | Synergistic |
| 1126:1 (2.5:1) | 1.04 (0.82, 1.27) | 0.95 (0.66, 1.24) | 0.87 (0.45, 1.29) | |
| Compound 3 | | | | |
| 111:1 (1:1) | 0.92 (0.77, 1.06) | 0.90 (0.70, 1.10) | 0.89 (0.58, 1.21) | Additive- |
| 44:1 (1:2.5) | 0.71 (0.60, 0.82) | 0.66 (0.51, 0.80) | 0.61 (0.40, 0.82) | Synergistic |
| 278:1 (2.5:1) | 0.41 (0.36, 0.47) | 0.41 (0.33, 0.48) | 0.40 (0.28, 0.52) | |

^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

### Pharmaceutical Composition and Methods of Use

Compound 1 inhibits HIV proviral integration, an essential step in HIV replication, and can be useful for the treatment of HIV infection and the consequent pathological conditions such as AIDS or ARC. As shown above, Compound 1 or its prodrug Compound 2 is active in conjunction with a wide variety of other agents and may be particularly beneficial in HAART and other new combination compositions and therapies.

Compound 1 or Compound 2 will generally be given as a pharmaceutical composition, and the active ingredient of the composition may be comprised of Compound 1 or Compound 2 alone or Compound 1 or Compound 2 and at least one other agent used for treating AIDS or HIV infection. The compositions will generally be made with a pharmaceutically accepted carrier or vehicle, and may contain conventional exipients. The compositions are made using common formulation techniques. The invention encompasses all conventional forms. Solid and liquid compositions are preferred. Some solid forms include powders, tablets, capsules, and lozenges. Tablets include chewable, buffered, and extended release. Capsules include enteric coated and extended release capsules. Powders are for both oral use and reconstitution into solution. Powders include lyophilized and flash-melt powders. In a solid composition, Compound 1 or Compound 2 and any antiretroviral agent are present in dosage unit ranges. Generally, Compound 1 or Compound 2 will be in a unit dosage range of 1-1000 mg/unit. Some examples of dosages are 1 mg, 10, mg, 100, mg, 250 mg, 500 mg, and 1000 mg. Generally, other antiretroviral agents will be present in a unit range similar to agents of that class used clinically. Typically, this 0.25-1000 mg/unit.

Liquids include aqueous solutions, syrups, elixers, emusions, and suspensions. In a liquid composition, Compound 1 or Compound 2 and any antiretroviral agent are present in dosage unit ranges. Generally, Compound 1 or Compound 2 will be in a unit dosage range of 1-100 mg/mL. Some examples of dosages are 1 mg/mL, 10 mg/mL, 25, mg/mL, 50 mg/mL, and 100 mg/mL. Generally, other antiretroviral agents will be present in a unit range similar to agents of that class used clinically. Typically, this is 1-100 mg/mL.

The invention encompasses all conventional modes of administration; oral and parenteral (injected intramuscular, intravenous, subcutanaeous) methods are preferred. Generally, the dosing regimen will be similar to other antiretroviral agents used clinically. Typically, the daily dose will be 1-100 mg/kg body weight daily for Compound 1 or Compound 2. Generally, more compound is required orally and less parenterally. The specific dosing regime, however, will be determined by a physician using sound medical judgement.

The invention also encompasses methods where Compound 1 or Compound 2 is given in combination therapy. That is, Compound 1 or Compound 2 can be used in conjunction with, but separately from, other agents useful in treating AIDS and HIV infection. Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, immunomodulators, and anti-infectives. In these combination methods, Compound 1 or Compound 2 will generally be given in a daily dose of 1-100 mg/kg body weight daily in conjunction with other agents. The other agents generally will be given in the amounts used therapeutically. The specific dosing regime, however, will be determined by a physician using sound medical judgement.

Table 7 lists some agents useful in treating AIDS and HIV infection, which are suitable for this invention. The invention, however, is not limited to these agents.

TABLE 7

| | | |
|---|---|---|
| DRUG NAME | MANUFACTURER | INDICATION |

ANTIVIRALS

| | | |
|---|---|---|
| 097 | Hoechst/Bayer | HIV infection, AIDS, |
| (non-nucleoside | | ARC |
| reverse | | |
| transcriptase | inhibitor) | |
| Amprenavir | Glaxo Wellcome | HIV infection, AIDS, |
| 141 W94 | | ARC |
| GW 141 | | |
| (protease inhibitor) | | |
| Abacavir (1592U89) | Glaxo Wellcome | HIV infection, AIDS, |
| GW 1592 | | ARC |
| (RT inhibitor) | | |
| Acemannan | Carrington Labs | ARC |
| | (Irving, TX) | |
| Acyclovir | Burroughs Wellcome | HIV infection, AIDS, |
| | | ARC, in combination |
| | | with AZT |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection, ARC, |
| AL-721 | Ethigen | PGL HIV positive, AIDS |
| | (Los Angeles, CA) | |
| Alpha Interferon | Glaxo Wellcome | Kaposi's sarcoma |
| HIV in combination | | |
| w/Retrovir | | |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha | (Rockville, MD) | |
| aberrant | | |
| Interferon | | |
| AR177 | Aronex Pharm | HIV infection, AIDS, |
| | | ARC |
| Beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| (protease inhibitor) | | |
| BMS-234475 | Bristol-Myers Squibb/ | HIV infection, AIDS, |
| (CGP-61755) | Novartis | ARC |
| (protease inhibitor) | | |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV peripheral, CMV |
| | | retinitis |
| Delaviridine | Pharmacia-Upjohn | HIV infection, AIDS, |
| (RT inhibitor) | | ARC |
| Dextran Sulfate | Ueno Fine Chem. | AIDS, ARC, HIV |
| | Ind. Ltd. (Osaka, | positive asymptomatic |
| | Japan) | |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combinationwith |
| | | AZT/d4T |
| DMP-450 | AVID | HIV infection, AIDS, |
| (protease inhibitor) | (Camden, NJ) | ARC |
| Efavirenz | DuPont Merck | HIV infection, AIDS, |
| (DMP 266) | | ARC |
| (—)6-Chloro-4-(S)- | | |
| cyclopropylethynyl- | | |
| 4(S)-trifluoro- | | |
| methyl-1,4-dihydro- | | |
| 2H-3,1-benzoxazin- | | |
| 2-one, STOCRINE | | |
| (non-nucleoside RT | | |
| inhibitor) | | |
| EL10 | Elan Corp, PLC | HIV infection |
| | (Gainesville, GA) | |
| Famciclovir | Smith Kline | herpes zoster, herpes |
| | | simplex |
| FTC | Emory University | HIV infection, AIDS, |
| (reverse | | ARC |
| transcriptase | | |
| inhibitor) | | |
| GS 840 | Gilead | HIV infection, AIDS, |
| (reverse | | ARC |
| transcriptase | | |
| inhibitor) | | |
| HBY097 | Hoechst Marion | HIV infection, AIDS, |
| (non-nucleoside | Roussel | ARC |
| reverse | | |
| transcriptaseinhibitor) | | |
| Hypericin | VIMRx Pharm. | HIV infection, AIDS, |
| | | ARC |
| Recombinant Human | Triton Biosciences | AIDS, Kaposi's sarcoma, |
| Interferon Beta | (Almeda, CA) | ARC |
| Interferon alfa-n3 | Interferon Sciences | ARC, AIDS |
| Indinavir | Merck | HIV infection, AIDS, |
| | | ARC, asymptomatic HIV |
| | | positive, also in |
| | | combination with |
| | | AZT/ddI/ddC |
| ISIS 2922 | ISIS Pharmaceuticals | CMV retinitis |
| KNI-272 | Nat'l Cancer Institute | HIV-associated diseases |
| Lamivudine, 3TC | Glaxo Wellcome | HIV infection, AIDS, |
| (reverse transcriptase | | ARC, also with AZT |
| inhibitor) | | |
| Lobucavir | Bristol-Myers Squibb | CMV infection |
| Nelfinavir | Agouron | HIV infection, AIDS, |
| (protease inhibitor) | Pharmaceuticals | ARC |
| Nevirapine | Boeheringer | HIV infection, AIDS, |
| (RT inhibitor) | Ingleheim | ARC |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, AIDS, |
| (protease inhibitor) | | ARC |
| Probucol | Vyrex | HIV infection, AIDS |
| RBC-CD4 | Sheffield Med. | HIV infection, AIDS, |
| | Tech (Houston, TX) | ARC |
| Ritonavir | Abbott | HIV infection, AIDS, |
| (protease inhibitor) | | ARC |
| Saquinavir | Hoffmann- | HIV infection, AIDS, |
| (protease inhibitor) | LaRoche | ARC |
| Stavudine; d4T | Bristol-Myers Squibb | HIV infection, AIDS, |
| Didehydrodeoxy- | | ARC |
| thymidine | | |
| Valaciclovir | Glaxo Wellcome | Genital HSV & |
| | | CMVinfections |
| Virazole | Viratek/ICN | asymptomatic HIV- |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's sarcoma, |
| | | in combination with |
| | | other therapies |
| Tenofovir disoproxil, | Gilead | HIV infection, AIDS |
| fumarate salt | | |
| (Viread ®) | | |
| (reverse transcriptase | | |
| inhibitor) | | |
| Combivir ® | GSK | HIV infection, AIDS |
| (reverse transcriptase | | |
| inhibitor) | | |
| abacavir succinate | GSK | HIV infection, AIDS |
| (or Ziagen ®) | | |
| (reverse transcriptase | | |
| inhibitor) | | |
| Reyataz ® | Bristol-Myers Squibb | HIV infection, AIDS |
| (atazanavir) | | |
| Fuzeon | Roche/Trimeris | HIV infection, AIDS, |
| (Enfuvirtide, T-20) | | viral fusion inhibitor |
| Trizivir ® | | HIV infection, AIDS |
| Kaletra ® | Abbott | HIV infection, AIDS, |
| | | ARC |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | Blocks HIV fusion with |
| | | CD4+ cells |
| Gamma Interferon | Genentech | ARC, in combination |
| | | w/TNF (tumor necrosis |
| | | factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, combination |
| Macrophage Colony | | w/AZT |
| Stimulating Factor | | |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in CD4 |
| Interleukin-2 | | cell counts |
| (aldeslukin) | | |
| Immune Globulin | Cutter Biological | Pediatric AIDS, in |
| intravenous | (Berkeley, CA) | combination w/AZT |
| (human) | | |
| IMREG-1 | Imreg | AIDS, Kaposi's sarcoma, |
| | (New Orleans, LA) | ARC, PGL |
| IMREG-2 | Imreg | AIDS, Kaposi's sarcoma, |
| | (New Orleans, LA) | ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma AIDS, |
| Muramyl-Tripeptide | Amgen | in combination w/AZT |
| Granulocyte | | |
| Colony Stimulating | | |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma, AIDS, |
| Alfa 2a | in combination w/AZT | ARC |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal meningitis, |
| | | candidiasis |
| Pastille | Squibb Corp. | Prevention of oral |
| Nystatin Pastille | | candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia assoc. |
| Erythropoietin | | with AZT therapy |
| Recombinant Human | Serono | AIDS-related wasting, |
| Growth Hormone | | cachexia |
| Megestrol Acetate | Bristol-Myers Squibb | Treatment of anorexia |
| | | assoc. W/AIDS |
| Testosterone | Alza, Smith Kline | AIDS-related wasting |
| Total Enteral | Norwich Eaton | Diarrhea and |
| Nutrition | Pharmaceuticals | malabsorption related to |
| | | AIDS |

1. A method for treating HIV infection in a human patient comprising administering a therapeutically effective amount of 3-[(4-fluorobenzyl)methoxycarbamoyl]-2-hydroxyacrylic acid or 2-(2,2)-dimethyl-5-oxo-[1,3]-dioxolan-4-ylidene)-N-(4-fluorobenzyl)-N-methoxyacetamide or a pharmaceutically acceptable salt or solvate thereof with a therapeutically effective amount of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

2. The method of claim 1 wherein the agent is a nucleoside HIV reverse transcriptase inhibitor.

3. The method of claim 2 wherein the nucleoside HIV reverse transcriptase inhibitor is selected from the group consisting of abacavir, didanosine, emtricitabine, lamivudine, stavudine, tenofovir, zalcitabine, and zidovudine, or a pharmaceutically acceptable salt or solvate thereof.

4. The method of claim 1 wherein the agent is a non-nucleoside HIV reverse transcriptase inhibitor.

5. The method of claim 4 wherein the non-nucleoside HIV reverse transcriptase inhibitor is selected from the group consisting of delavirdine, efavirenz, and nevirapine, or a pharmaceutically acceptable salt or solvate thereof.

6. The method of claim 1 wherein the agent is an HIV protease inhibitor.

7. The method of claim 6 wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

8. The method of claim 1 wherein the agent is an HIV fusion inhibitor.

9. The method of claim 8 wherein the HIV fusion inhibitor is enfuvirtide or T-1249, or a pharmaceutically acceptable salt or solvate thereof.

10. The method of claim 1 wherein the agent is an HIV attachment inhibitor.

11. The method of claim 10 where the HIV attachment inhibitor is Compound 3.

12. The method of claim 1 wherein the agent is a CCR5 inhibitor.

13. The method of claim 12 wherein the CCR5 inhibitor is selected from the group consisting of Sch-C, Sch-D, TAK-220, PRO-140, and UK-427,857, or a pharmaceutically acceptable salt or solvate thereof.

14. The method of claim 1 wherein the agent is a CXCR4 inhibitor.

15. The method of claim 14 wherein the CXCR4 inhibitor is AMD-3 100, or a pharmaceutically acceptable salt or solvate thereof.

16. The method of claim 1 wherein the agent is an HIV budding or maturation inhibitor.

17. The method of claim 16 wherein the budding or maturation inhibitor is PA-457, or a pharmaceutically acceptable salt or solvate thereof.

18. The method of claim 1 wherein the agent is an HIV integrase inhibitor.

19. The method of claim 18 wherein the HIV integrase inhibitor is C-2507 or its analogs, L-870810 or its analogs, L-870812 or its analogs, 1380 or its analogs, and JTK-303, or a pharmaceutically acceptable salt or solvate thereof.

20. A pharmaceutical composition useful for treating AIDS or HIV infection comprising a therapeutically effective amount of 3-[(4-fluorobenzyl)methoxycarbamoyl]-2-hydroxyacrylic acid or 2-(2,2)-dimethyl-5-oxo-[1,3]-dioxolan-4-ylidene)-N-(4-fluorobenzyl)-N-methoxyacetamide, a pharmaceutically acceptable salt, or solvate thereof with at least one other agent used for treatment of AIDS, or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

21. The composition of claim 20 wherein the agent is a nucleoside HIV reverse transcriptase inhibitor.

22. The composition of claim 21 wherein the nucleoside HIV transcriptase inhibitor is selected from the group consisting of abacavir, didanosine, emtricitabine, lamivudine, stavudine, tenofovir, zalcitabine, and zidovudine, or a pharmaceutically acceptable salt or solvate thereof.

23. The composition of claim 20 wherein the agent is a non-nucleoside HIV reverse transcriptase inhibitor.

24. The composition of claim 23 wherein the non-nucleoside HIV reverse transcriptase inhibitor is selected from the group consisting of delavirdine, efavirenz, and nevirapine, or a pharmaceutically acceptable salt or solvate thereof.

25. The composition of claim 20 wherein the agent is an HIV protease inhibitor.

26. The composition of claim 25 wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

27. The composition of claim 20 wherein the agent is an HIV fusion inhibitor.

28. The composition of claim 27 wherein the HIV fusion inhibitor is enfuvirtide or T-1249, or a pharmaceutically acceptable salt or solvate thereof.

29. The composition of claim 20 wherein the agent is an HIV attachment inhibitor.

30. The composition of claim 29 where the HIV attachment inhibitor is Compound 3.

31. The composition of claim 20 wherein the agent is a CCR5 inhibitor.

32. The composition of claim 31 wherein the CCR5 inhibitor is selected from the group consisting of Sch-C, Sch-D, TAK-220, PRO-140, and UK-427,857, or a pharmaceutically acceptable salt or solvate thereof.

33. The composition of claim 20 wherein the agent is a CXCR4 inhibitor.

34. The composition of claim 33 wherein the CXCR4 inhibitor is AMD-3 100, or its analogs, or a pharmaceutically acceptable salt or solvate thereof.

35. The composition of claim 20 wherein the agent is an HIV budding or maturation inhibitor.

36. The composition of claim 35 wherein the budding or maturation inhibitor is PA-457, or a pharmaceutically acceptable salt or solvate thereof.

37. The composition of claim 20 wherein the agent is an HIV integrase inhibitor.

38. The composition of claim 37 wherein the HIV integrase inhibitor C-2507 or its analogs, L-870810 or its analogs, L-870812 or its analogs, 1380 or its analogs, and JTK-303.
